(19) United States
(12) Patent Application Publication
(10) Pub. No.: US 2009/0029944 A1
(43) Pub. Date: Jan. 29, 2009

(54) METHODS FOR ADDING FATTY ACIDS TO AGENTS IN AQUEOUS SOLUTION TO IMPROVE BIOAVAILABILITY

(76) Inventor: Keith K. Skinner, Aurora, CO (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/142,698

(22) Filed: Jun. 19, 2008

Related U.S. Application Data
(60) Provisional application No. 60/945,014, filed on Jun. 19, 2007.

(51) Int. Cl. A61K 31/715; A61K 31/352; A61K 31/12; A61K 31/05

(52) U.S. Cl. 514/62; 514/456; 514/683; 514/733

(57) ABSTRACT

Adding fatty acids to agents enhances deliverability of the antioxidant, making the agent less likely to be degraded prior to intercellular delivery. Additionally, adding fatty acids to agents provides a time lapse-type mechanism to the agents. Fatty acid modified agents are made with a novel process in aqueous solution, reducing the need to use organic solvents. Agents that can be modified as disclosed herein include nearly any biological molecule that have hydroxyl, amine, or sulphydryl sites, including antioxidants, such as glutathione and polyphenols (e.g., ECGC, curcumin, or resveratol), saccharides, such as glucosamine, and other glycans.

### RELATED APPLICATIONS

The present disclosure claims the Paris Convention Priority of U.S. Provisional Patent Application Ser. No. 60/945,014, the contents of which are incorporated by reference as if disclosed in the present disclosure.

### BACKGROUND

This disclosure relates to make a modified agent by adding fatty acids to make the agents more readily deliverable intracellularly.

### SUMMARY

Adding fatty acids to agents enhances deliverability of the antioxidant, making the agent less likely to be degraded prior to intercellular delivery. Additionally, adding fatty acids to agents provides a time lapse-type mechanism to the agents. Fatty acid modified agents are made with a novel process in aqueous solution, reducing the need to use organic solvents. Agents that can be modified as disclosed herein include nearly any biological molecule that have hydroxyl, amine, or sulphydryl sites, including antioxidants, such as glutathione and polyphenols (e.g., ECGC, curcumin, or resveratol), saccharides, such as glucosamine, and other glycans.

According to a feature of the present disclosure, a method is disclosed comprising suspending an agent in aqueous solution, adding a fatty acid chloride source to the aqueous solution and base source to make the aqueous solution basic, stirring, purifying the resulting precipitate.

According to a feature of the present disclosure, a composition is disclosed comprising a therapeutically effective amount of an agent having at least one fatty acid molecule bonded thereto, wherein each fatty acid molecule protects the agent thereby improving the agent's bioavailability, and a pharmaceutically acceptable carrier.

According to a feature of the present disclosure, a composition is disclosed comprising a food product having suspended therein an agent having at least one fatty acid molecule bonded thereto, wherein each fatty acid molecule protects the agent to improve the bioavailability of the agent.

### DRAWINGS

The above-mentioned features and objects of the present disclosure will become more apparent with reference to the following description taken in conjunction with the accompanying drawing:

FIG. 1 is block diagram of an embodiment of a process for creating fatty acid modified agents according to the present disclosure.

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction (inclusive of the term “and”) and shall not indicate an exclusive disjunction unless expressly indicated as such or notated as “xor.”

The present disclosure discloses a method for modifying agents with lipids thereby making the agents more readily bioavailable. Moreover, adding fatty acids to agents further allows the skin to more easily and efficiently absorb the agent through skin. In effect, adding fatty acids to agents creates both a vehicle for delivery through lipid bilayers of cells and the skin, and a “time release” effect as the agent is not bioavailable until the lipid side chains of the modified agent are cleaved. Fatty acids are cleaved carbon by carbon. Thus, agents having longer fatty acids bonded therefore take longer to become bioavailable than those having shorter fatty acid chains.

In recent years, antioxidants, including polyphenols, have been touted for their health benefits. Antioxidants are reducing agents that behave by acting to prevent oxidation by free radicals. Oxidation reactions, often induced by free radicals such as O₂ are critical for life. However, uncontrolled, free radicals pose a threat to cells because uninhibited they tend to form oxidative chain reactions and that interrupt the normal function of many biological pathways or deactivate biologically active molecules. Moreover, some free radicals, such as superoxide O₂— are toxic to cells and must be neutralized.

The present disclosure proposes a novel method of making agents more deliverable to cells by adding fatty acids to active sites on the agents. For example, the fatty acids are covalently bonded to one or more active sites of an antioxidant thereby preventing the antioxidant from reducing free radicals prior to delivery of the antioxidant at the target location. Consequently, antioxidants ingested orally, for example, are much less likely to reduce free radicals in the digestive tract, which means more antioxidant is delivered to the target location.

Artisans will recognize the agents that may benefit from the methods of the present disclosure. Agents have an active site that can reversibly react with the carboxyl end of fatty acids; these may include NH₂, SH₂, and OH sites. Indeed, the sites are preferentially bound amino or any free binding site, then sulphydryl or any free binding site, and finally hydroxyl. As will readily be recognized by artisans, NH₂ sites are will be modified first due their positive charge.

However, modification of the hydroxyl active sites is advantageous because ether bonds form between the fatty acid and the agent. The ether bonds are more stable in biologic systems, which means that the cell takes longer to break down the fatty acid and expose the active site and thereby allow the agent to have its intended affect.

According to embodiments, agents that may be modified according to the present disclosure include, but are not limited to, antioxidants, for example glutathione and glutathione variants; vitamins A (and other caretenes), C, and E; green tea extracts; hyaluronic acid; thioredoxin and thioredoxin reductase; superoxide dimutase; melatonin; uric acid; ubiquinone; lipoic acid, proanthocyanadins, and many others that would readily be recognized by artisans as having sites modifiable according to the teachings of the present disclosures by a person of ordinary skill in the art.

Polyphenols, including flavonols, catechins, and catechin gallates are modified with fatty acids, according to embodiments. For example, the following molecules are expressly contemplated as exemplary species of the genus of antioxidants and polyphenols according to embodiments disclosed herein: curcumin, demthoxycurcumin, bis-demethyloxycurcumin, resveratrol, trans-resveratrol, cis-resveratrol, epigallocatechin gallate (EGCG), gallocatechin, epigallocatechin, catechin gallate, epigallocatechin digallate, quercetin, kaempferol, myricitin, piceid, including both trans- and cis-piceid.

According to embodiments, saccharides or polysaccharides are modified with fatty acids to improve their bioavailability, for example, glucosamine, and other glycans, for example, chondroitin sulfate.

As well known to artisans, fatty acids comprise an aliphatic chain coupled to a carboxylic acid. According to embodiments, the carboxy end of fatty acids are reacted to the active sites of agents. The fatty acid-agent complex serves two purposes. First, the fatty acids reversibly block the active sites of the agent until the agent is delivered intracellularly. Second, the lipophilic aliphatic side chain or chains of the fatty acids allow the agent to more readily cross the cell membrane and penetrate skin, for example. Thus, by coupling agents and fatty acids, a more potent and effective delivery vehicle for the agents is introduced.

According to embodiments, any fatty acid having 2 or more carbons in the aliphatic chain are suitable to be coupled to agents. The fatty acids may be saturated or unsaturated. According to embodiments, butanoic acid (C4:0), pantanoid acid (C5:0), hexanoic acid (C6:0), octanoic acid (C8:0), nananoic acid (C9:0), decanoic acid (C10:0), dodecanoic acid (C12:0), tetradecanoic acid (C14:0), hexadecanoic acid (C16:0), heptadecanoic acid (C17:0), octadecanoic acid (C18:0), icosanoic acid (C20:0), docosanoic acid (C22:0), tetracosanoic acid (C24:0), hexacosanoic acid (C26:0), heptacosanoic acid (C27:0), octasonoic acid (C28:0), triacontanoic acid (C30:0), dotriacontanoic acid (C30:0), dotriacontanoic acid (C32:0), dotriacontanoic acid (C32:0), tritriacontanoic acid (C33:0), tetratriacontanoic acid (C34:0), or pentatriacontanoic acid (C35:0) are saturated fatty acids that are readily available and that are appropriate for use with the present disclosure. Fatty acids having more than 35 carbons and fatty acids having aliphatic chains of both an even and an odd number of carbons are equally applicable with the teachings of the present disclosure.

Similarly, unsaturated fatty acids having any number of double or triple bonds in both a-cis or -trans configurations are expressly contemplated. For example, myristoleic acid (C14:1), palmitoleic acid (C16:1), oleic acid (C18:1), Linoleic acid (C18:2), a-linoleic acid (C18:3), arachidonic acid (C20:4), eicosapentaenoic acid (C20:4), Eicosapentaenoic acid (C20:5), Erucic Acid (C22:1), or docosahexaenoic acid (C22:6) are examples of common unsaturated fatty acids that may be coupled to agents according to the present disclosure. Other unsaturated fatty acids are expressly contemplated, as would be known to artisans.

Moreover, according to embodiments, the fatty acids of the present disclosure may be oils, such as olive oil, jojoba oil, sunflower oil, safflower oil, rapeseed oil, corn oil, soya oil, wheat germ oil, cottonseed oil, almond oil or oils of other nuts, palm oil, coconut oil, vegetable oil, butter, lard, as well as other oils comprising, at least in part, fatty acids. Obviously, where the agent is to be delivered intracellularly, the oil or fatty acid must be non-toxic.

According to embodiments, the oil selected my comprise oils known to be healthy, such as olive oil or omega-3 fatty acids. Use of such health-type oils may be of interest to the health food markets, etc. Moreover, according to embodiments the agents may comprise health food supplements to be sold as such or may be included as additives in containers of oil purchased, for example, at the grocery story for general cooking or spread purposes.

Once delivered intracellularly, enzymes within the cell cleave off the fatty acids, allowing the bioactive sites of the agent to become available. Cleaving of the fatty acids occurs carbon by carbon. Consequently, the longer the aliphatic chain of the fatty acid, the longer the agent will be protected by the fatty acid(s). Indeed, by using multiple oils having different size aliphatic chains, a time release-like product is created whereby the agents having the shorter aliphatic chains become bioavailable more quickly on average than those having longer aliphatic chains.

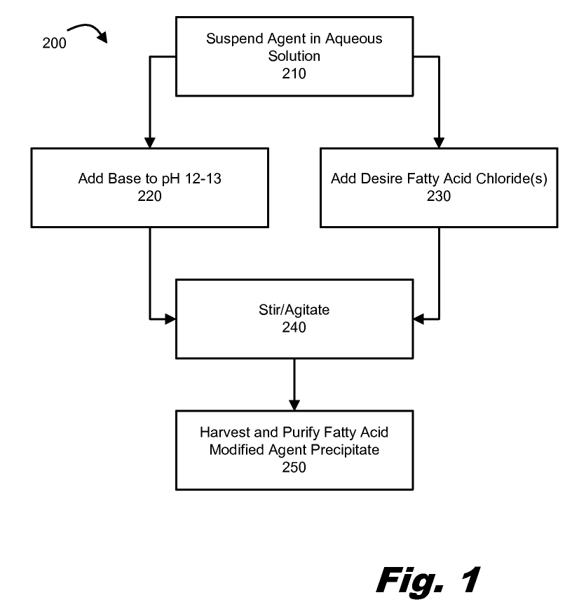

The process for protecting agents with fatty acids is performed in aqueous solution using the fatty acid chloride of the fatty acids being used to modify, as illustrated according to an embodiment shown in FIG. 1. The process 200 may be performed in quantities of scale without appreciable modification in the core steps of the procedure. Initially, the agent of interest is dissolved into water in operation 210. According to embodiments, the concentration of the agent in the water is increased to a maximum concentration.

After the agent to be modified is dissolved into water, the pH is raised to pH 12-13 with a base, according to operation 220. According to embodiments, the base is an inorganic base, such as NaOH, which prevent undesirable side reactions. Throughout the modification process, the pH is kept in the range of pH 12-13 to drive the modification reaction. After the pH is raised to pH 12-13, the fatty acid chloride is added to drop-wise to the solution in operation 230 under agitating/stirring in operation 240, together with additional base to maintain the desired pH (operation 220). As the fatty acid chloride is added to each agent, the resulting product falls out of solution as a precipitate. According to similar embodiments, the solution need not have the pH raised before adding the fatty acid chloride and the base, whereby the pH will be raised as a matter of course during the reaction.

The precipitate is then harvested and purified in operation 250. Harvesting may occur simply by decanting the water, washing the precipitate with water at least once, and drying. The resultant dry precipitate is the agent coupled to one or more fatty acid molecules. The precipitate may then be added as an additive to other products such as vitamin tablets, lotion, etc. for delivery purposes. According to embodiments, fatty acid modified agent products by the disclosed process are expressly contemplated.

It will be understood by artisans that the methods of the instant disclosure may be performed on a large scale without appreciable changes to the principles disclosed by the exemplary protocol.

According to embodiments, the fatty acid modified agent products may be further modified, either before or after the process disclosed herein to provide further desirable characteristics. For example, agent molecules, such as glutathione, may be esterified prior to the process disclosed herein. Other similar modifications that are known in the art, such as acetylation with glutathione, are also possible and expressly contemplated, provided active sites on the agent are available for modification.

According to embodiments, the fatty acid modified agents are included in a pharmaceutical, nutraceutical, or cosmeceutical composition together with additional active agents, carriers, vehicles, excipients, or auxiliary agents identifiable by a person skilled in the art upon reading of the present disclosure.

The pharmaceutical, nutraceutical, or cosmeceutical compositions comprise at least one pharmaceutically acceptable carrier. In such pharmaceutical, nutraceutical, or cosmeceutical compositions, the fatty acid modified agent forms the “active compound,” also referred to as the “active agent.” The term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical, nutraceutical, or cosmeceutical compositions are formulated to be compatible with their intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; agents such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

The term “subject” refers to humans and non-human primates (e.g., guerilla, macaque, marmoset), livestock animals (e.g., sheep, cow, horse, donkey, and pig), companion animals (e.g., dog, cat), laboratory test animals (e.g., mouse, rabbit, rat, guinea pig, hamster), captive wild animals (e.g., fox, deer), and any other organisms that will benefit from the agents of the present disclosure. There is no limitation on the type of animal that may benefit from the presently described agents. A subject, irrespective of whether it is a human or non-human organism, may be referred to as a patient, individual, animal, host, or recipient.

Pharmaceutical, nutraceutical, or cosmeceutical compositions suitable for an injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), or suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying or freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches, or the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

According to embodiments, administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, lotions, salves, gels, or creams as generally known in the art. The compounds can also be prepared in the form of suppositories (e.g., with conventional suppository bases such as cocoa butter and other glycerides) or retention enemas for rectal delivery.

According to embodiments and in addition to the modification of the fatty acid modified agents disclosed herein, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, which is incorporated by reference herein.

According to embodiments, it is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are believed to be most effective. While compounds that exhibit toxic side effects can be used, care should be taken to design a delivery system that targets such compounds to the site of affected location to minimize potential damage to uninfected cells and, thereby, reduce side effects.

The data obtained from cell culture assays and animal studies can be used in formulating a range of dosage for use in humans, according to embodiments. The dosage of such compounds lies preferably within a range of circulating concentrations that include the ED₅₀ with little or no toxicity. The dosage can vary within this range depending upon the dosage form employed and the route of administration utilized. For any compound used according the compositions and methods of the present disclosure, the therapeutically effective dose can be estimated initially from cell culture assays, according to embodiments. A dose can be formulated in animal models to achieve a circulating plasma concentration range that includes the IC₅₀ (i.e., the concentration of the test compound which achieves a half-maximal inhibition of symptoms) as determined in cell culture, according to embodiments. Such information can be used to more accurately determine useful doses in humans. Levels in plasma may be measured, for example, by high performance liquid chromatography.

As defined herein, a therapeutically effective amount of the active compound (i.e., an effective dosage) may range from about 0.001 to 100 g/kg body weight, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

According to embodiments, a kit of parts perform at least one of the methods herein disclosed, the kit of parts comprising at least one agent to be modified by a fatty acid together with at least one fatty acid chloride for creating fatty acid modified agents. According to embodiments, a kit of parts comprises fatty acid chlorides and other reagents, but not the agent to be modified.

According to embodiments, the kits include compositions comprising active agents other than the agents to be modified with the fatty acids or the fatty acid chlorides, identifiers of a biological event, or other compounds identifiable by a person skilled upon reading of the present disclosure. The term “identifier” refers to a molecule, metabolite or other compound, such as antibodies, DNA or RNA oligonucleotides, able to discover or determine the existence, presence, or fact of or otherwise detect a biological event under procedures identifiable by a person skilled in the art; exemplary identifiers are antibodies, exemplary procedures are western blot, nitrite assay and RT-PCR, or other procedures as described in the Examples.

The kit also comprises, according to embodiments, at least one composition comprising an effective amount of fatty acid chlorides and a cell line that produces an agent of choice. The compositions and the cell line of the kits of parts to be used to perform the at least one method herein disclosed according to procedure identifiable by a person skilled in the art.

### EXAMPLE 1

The methods of the present disclosure may be used to make a modified glutathione molecule. Glutathione is a potent antioxidant having three primary active sites: the carboxy and amino terminal ends of the peptide sequence, as well as the sulphydryl residue of the cysteine amino acid. According to embodiments, unmodified glutathione or previously esterified glutathione is dissolved into water. Sodium hydroxide is added to bring the pH of the solution to pH 12-13. The solution is constantly stirred or agitated while a solution containing a palmitic acid chloride is added drop-wise into the water-glutathione solution. Concurrently, additional sodium hydroxide is added to the solution to maintain the pH at between 12-13. Under these conditions, the palmitic acid reacts with the active sites of the glutathione and the modified glutathione falls out of solution. Artisans will recognize that the palmitic acids reacts first with the amino residue, followed by the sulphyrdel residual, and then finally the carboxyl residue.

The reaction is propagated until an efficient yield of modified glutathione is precipitated. Thereafter, the water from the glutathione solution is decanted, whereby all unreacted fatty acid and glutathione is removed. The precipitate is washed one or more times to remove residual unreacted fatty acid and glutathione, as well as to decrease the pH to physiologically acceptable levels. After washing, the precipitate is dried.

Thereafter, the precipitate may added to lotions or vitamins, for example. The modified palmitated glutathione is a better deliverable because the fatty acid makes the glutathione molecule more readily absorbed through the skin or cell membrane permeable. Moreover, the palmitate protects the glutathione in transit until the fatty acid is fully cleaved from the glutathione molecule.

### EXAMPLE 2

Similarly, the procedure of Example 1 is duplicated. However, rather than using palmitic acid as the fatty acid, olive or jojoba oil chlorides are added as the fatty acid chloride. Artisans will readily recognize and understand the process of making the olive or jojoba oil chloride. The resulting olive oil-glutathione or jojoba oil-glutathione may then be marketed in health food stores as a food product having an agent therein modified with fatty acids to be used in cooking or other desirable applications.

### EXAMPLE 3

Oils that have multiple fatty acids, each having different sized aliphatic chains may be used to create “time-release” agents. Shorter aliphatic chains are cleaved more quickly to expose the active site of agents, while the longer aliphatic chains are protected for longer. Thus, the net effect is an extended delivery time for the modified agents.

### EXAMPLE 4

Fatty acid chlorides may also be added to antioxidants with a majority of hydroxyl sites such as carnatine and hyaluronic acid. Each of these antioxidants have a plurality of hyroxyl sites. The fatty acid chlorides are added to the hydroxyl active sites, as disclosed herein or as commonly known in the art. To create a time-release-type effect, modifications of varying the size of the attached fatty acids or as otherwise disclosed herein or known and understood by artisans according to the principles disclosed herein is desirable, which releases the active site over a wider range of time. The fatty acid modified antioxidants may be created according to the method of Example 1 or as otherwise disclosed herein or known and understood by artisans according to the principles disclosed herein, according to embodiments.

### EXAMPLE 5

The fatty acid chlorides are combined with polyphenols to create fatty acid modified polyphenols. For example, EGCG, resveratrol, curcumin, and related derivates to them. The resulting fatty acid modified has increased bioavailability as compared to the non-modified polyphenols. To create a time-release-type effect, modifications of varying the size of the attached fatty acids or as otherwise disclosed herein or known and understood by artisans according to the principles disclosed herein is desirable, which releases the active site over a wider range of time. The fatty acid modified polyphenols may be created according to the method of Example 1, according to embodiments or as otherwise disclosed herein or known and understood by artisans according to the principles disclosed herein.

### EXAMPLE 6

Fatty acid chlorides are added to glucosamine or chondroitin sulfate to form at least one of fatty acid modified glucosamine or fatty acid modified chondroitin sulfate that have increased bioavailable when compared to non-modified glucosamine or chondroitin sulfate. To create a time-release-type effect, modifications of varying the size of the attached fatty acids or as otherwise disclosed herein or known and understood by artisans according to the principles disclosed herein is desirable, which releases the active site over a wider range of time. The fatty acid modified glycans may be created according to the method of Example 1, according to embodiments or as otherwise disclosed herein or known and understood by artisans according to the principles disclosed herein.

While the compositions, kits, devices, and methods have been described in terms of what are presently considered to be the best embodiments now known, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
suspending an agent in aqueous solution;
adding a fatty acid chloride source to the aqueous solution and base source to make the aqueous solution basic;
stirring;
purifying the resulting precipitate.

2. The method of claim 1, wherein the reaction is performed at pH 12-13.

3. The method of claim 1, wherein the aliphatic chain of the fatty acid chloride contains from 4-22 carbons.

4. The method of claim 1, wherein the fatty acid chloride source comprises a plurality of fatty acids chlorides having varied aliphatic chain lengths.

5. The method of claim 1, wherein the fatty acid chloride source comprises an oil.

6. The method of claim 5, wherein the oil is one of olive oil, almond oil, or jojoba oil.

7. The method of claim 1, wherein purification further comprises decanting the solution, washing the precipitate at least once, and drying the precipitate.

8. The method of claim 1, wherein the product is delivered orally.

9. The method of claim 1, wherein the product is delivered topically.

10. The method of claim 1, wherein the agent comprises an antioxidant.

11. The method of claim 1, wherein the agent comprises a polyphenol.

12. The method of claim 11, wherein the polyphenol is at least one of epigallocatechin, curcumin, or resveratrol.

13. The method of claim 1, wherein the agent is glucosamine.

14. A product by the process of claim 1.

15. A composition comprising:
a therapeutically effective amount of an agent having at least one fatty acid molecule bonded thereto, wherein each fatty acid molecule protects the agent thereby improving the agent's bioavailability; and
a pharmaceutically acceptable carrier.

16. The composition of claim 15, wherein the agent is an antioxidant.

17. The composition of claim 15, wherein the agent is a polyphenol.

18. The composition of claim 17, wherein the polyphenol is at least one of epigallocatechin, curcumin, or resveratrol.

19. The composition of claim 15, wherein the agent is glucosamine.

20. The composition of claim 15, wherein the fatty acid molecules bound to the therapeutically effective amount of an agent have differentially sized aliphatic chains.

21. A composition comprising:
a food product having suspended therein an agent having at least one fatty acid molecule bonded thereto, wherein each fatty acid molecule protects the agent to improve the bioavailability of the agent.

22. The composition of claim 21, wherein the food product comprises at least one of an oil.
